(19) United States
(12) Patent Application Publication
(10) Pub. No.: US 2014/0330059 A1
(43) Pub. Date: Nov. 6, 2014

(54) METHOD OF USING CARBON NANOTUBES FUEL PRODUCTION

(71) Applicant: Board of Trustees, Southern Illinois University, Carbondale, IL (US)

(72) Inventors: Saikat Talapatra, Carbondale, IL (US); Kanchan Mondal, Carbondale, IL (US)

(73) Assignee: Board of Trustees, Southern Illinois University, Carbondale, IL (US)

(21) Appl. No.: 14/209,272

(22) Filed: Mar. 13, 2014

Related U.S. Application Data
(60) Provisional application No. 61/788,669, filed on Mar. 15, 2013.

(51) Int. Cl. C07C 1/04; C07C 2/00

(52) U.S. Cl. CPC C07C 1/0425; C07C 2/00
USPC 585/310; 422/630

(57) ABSTRACT

Use of a particular form of carbon—carbon nanotubes—as the catalyst. The present technology uses CNTs produced via air-assisted chemical vapor deposition (CVD), which can produce these extremely active catalysts (over an order higher in the magnitude of the activity of the catalyst as compared to the conventional Fe based catalyst) in a single step for converting carbonaceous materials such as coal, biomass, natural gas to liquid fuels such as gasoline, diesel, jet fuel, kerosene etc. . . . , which is generally accomplished through three process blocks.

### CROSS-REFERENCE TO RELATED APPLICATIONS

This application claims the benefit of and priority to U.S. Provisional Application Ser. No. 61/788,669, Filed Mar. 15, 2013 and entitled CARBON NANOTUBES and is incorporated herein in its entirety.

### BACKGROUND

1. Field

The subject matter of this application relates to the production of carbon fuels and more particularly relates to use of carbon nanotubes for fuel production.

2. Background Art

Clean, domestic energy sources have garnered increasing interest. One such process involves the direct and indirect liquefaction of coal to make liquid fuels and the precursors to several chemicals. The Fischer-Tropsch (FT) synthesis process is a catalyzed chemical reaction that converts a mixture of carbon monoxide and hydrogen gas, the synthesis (syn) gas produced during coal gasification, into a range of straight chained and branched olefins, paraffins, and oxygenates. When this process is combined with suitable coal gasification technologies upstream and a refining process downstream, gasoline and diesel fuels with low trace and sulfur components but with high octane and cetane numbers can be produced.

In the Fischer Tropsch synthesis reaction, CO and H2 react to form methyl radicals that combine to form hydrocarbons. The FT synthesis process consists of five basic steps: a) reactant adsorption, b) chain initiation, c) chain growth, d) chain termination, and e) product desorption. Water is a byproduct of the primary FT synthesis reaction. However, carbon dioxide may be a byproduct of the primary FT reaction if the availability of hydrogen at the reactions site is low. Thus the hydrogen spillover onto the support and its ability to weakly adsorb these hydrogen moieties is a determining factor for the carbon efficiency of this reaction.

A catalyst is typically dispersed on a catalytic support. These supports allow for a greater catalytic surface area to be exposed thereby enhancing the reaction rate. However, in the FT synthesis, the reaction efficiency depends on both the activity of the metal catalyst and the type of catalyst support. The two primary active catalysts are cobalt and iron, typically prepared over a variety of support materials such as γ-alumina, α-alumina, titania, and zirconia, usually in the form of oxides. Zirconia has a significant disadvantage as an FT catalyst because it possesses a small specific surface area and average pore size; making adequate deposition of the catalyst difficult. As a result, higher amounts of catalyst are required to achieve the same yield as alumina or silica. Silica and alumina supports have their drawbacks—these supports react with iron catalysts forming iron silicates and iron aluminates. These compounds are not active catalysts and use results in the loss of estimated catalyst activity. In addition, for cases where there is a strong support-catalyst interaction (such as in γ-alumina), the reduction of the catalyst to their metallic form (such that they are active towards FT synthesis reactions) is greatly hindered and requires higher temperatures and time. Higher temperatures not only mean higher energy demand for catalyst preparation but may actually result in the sintering of the catalysts causing it to deactivate.

A primary cause of catalyst deactivation is the highly exothermic nature of the FT reaction. The ceramic supports are generally not able to efficiently remove the heat from the catalyst surface resulting in hot spots and eventual deactivation of the catalyst. In the past, activated carbon (AC) has been used as non-oxide based FT catalyst support. Although the AC's have very high surface areas (˜1800 m²/g), most of the available surface area resides in pores with pore sizes less than 2 nm. These small pores limit the accessibility of the catalyst to the major portion of the surface areas offered by the AC. Furthermore, the micro porous texture of these materials often makes the metal catalyst aggregate on the outer surface during deposition.

In view of these major drawbacks of these supports (oxides and AC's) used in FT synthesis, alternative supports have been developed that have better surface, mechanical, and thermal properties. One material showing promise is the carbon nanotube (CNT). CNTs are carbon tubules with unique properties analogous to an ideal graphite fiber. A CNT is a graphene sheet, a single atom thick sheet of graphite with a honeycomb lattice, that is rolled upon itself. These CNTs have diameters in the nanometer range and lengths up to several micrometers. CNTs can be generally classified as single walled carbon nanotubes (SWNTs) or multi walled carbon nanotubes (MWNTs), depending on whether they are composed of a single graphene sheet or more than one graphene sheet, respectively. Both SWNTs and MWNTs offer advantages over conventional supports in terms of their large available specific surface area (50 m²/g-1600 m²/g), their mechanical strength, their chemical stability, and their excellent heat conductivities (thereby reducing hot spots on the active catalyst.)

CNTs are produced through a variety of methods including: arc discharge, laser ablation, and chemical vapor deposition. (Endo, M. et. al., Pure Appl. Chem., 78, 1703-13 (2006.)) Chemical vapor deposition is widely considered to be the ideal method for large scale production of high-quality CNTs. (Endo, M. et. al., Pure Appl. Chem., 78, 1703-13 (2006.)) Most importantly, CVD produces high purity nanotubes by in-situ etching of any amorphous carbon during the growth process. Hydrocarbon precursors for CVD are typically xylene, ethylene, or acetylene. CVD is a catalyzed reaction with an organometallic, typically ferrocene, used as the catalyst. In the vapor phase catalyst delivery method of CVD the catalyst and precursor are vaporized and flown over a substrate. This substrate is kept inside a temperature controlled furnace that is carefully tuned to maintain a temperature such that the catalyst particles precipitate on the substrate while the carbon atoms disassociate from the precursor. These carbon atoms stick to the catalyst particles and form carbon chains.

CVD is a promising method for growing carbon nanotubes; growth rates can reach up to 11 mm s⁻¹ for single-walled CNTs (with growth up to 4 cm) and multi-walled nanotubes can grow at an approximate rate of 1 cm hr⁻¹. (Zheng et. al., Natur. Mater., 3 673-6 (2004). Hong et. al, J. Am. Chem. Soc., 127 15336-7 (2005).) However, the growth rate of highly aligned CNTs is several orders of magnitude smaller. Several factors have been attributed to these slow growth rates: blocking of the carbon source by the nanotubes as growth progresses or deactivation of the catalyst due to encapsulation by amorphous carbon. (Li et al., Nanotechnology 19, 455609-26 (2008)). In cases where there is unimpeded flow of the carbon source through the nanotube forest, air assisted vapor phase delivery of the catalyst can prevent encapsulation of the catalyst particles by amorphous carbon. (Ibid) Adding oxidizers, such as air and water, to the CVD furnace allow for faster growth of aligned CNTs and prevent termination of CNT growth due to catalyst encapsulation, resulting in longer aligned CNTs.

Recent advances in CVD technology have enabled production of large quantities of CNTs using a relatively simple single-step production method. CNTs were produced using ferrocene as both the catalyst and the carbon precursor with a sulfur additive. (Zhu, H. and Wei, B., Chem. Commun., 3042-3044 (2007).) The sulfur additive's purpose is to promote growth of single-walled CNTs. (Ibid.) Multi-walled CNTs can also be produced using this method with different furnace temperatures and conditions. (Ibid.) However, SWNTs were produced by carefully calibrating the reaction chamber and furnace mechanism. The method of producing these SWNT comprises: adding a 10:1 molar ratio of ferrocene:sulfur to one end of the reaction tube; a steady flow of argon gas is applied to the volatile ferrocene mixture; a furnace heated to around 1100 C is placed around the ferrocene mixture; and then a argon and hydrogen flow gas mixture is introduced into the reaction chamber allowing for SWNT formation. (Ibid.) This method allows for large-scale formation of SWNT films on relatively irregular substrates. (Ibid)

U.S. Pat. No. 6,713,519 discloses a carbon nanotube-based FT catalyst comprising: a porous substrate, carbon nanotubes attached to the substrate, and catalysts adsorbed onto the CNTs. Alternately, a catalyst layer can be attached top on of the carbon nanotube layer so that the catalyst layer is surface exposed. In yet another embodiment, the catalyst layer is deposited on top of the CNTs. Despite the recognition that the catalysts supported on CNTs have superior properties when used as FT catalysts, this is attributed to the heat and mass transfer properties and surface area of the CNTs.

U.S. Pat. No. 7,288,576 is a continuing application of U.S. Pat. No. 6,713,519 disclosing the same catalyst but further claiming a method of utilizing these catalysts in a reactor chamber or micro-channel reactor.

U.S. Patent Published Application number 2008/0176740 discloses a method of producing catalysts containing metal-loaded carbon nanotubes. The CNTs act as a support for the metal catalyst. These nanotubes can be functionalized and loaded with metal catalysts before or after linking them into an aggregated carbon nanotube structure. Loading methods for the catalysts onto the nanotubes include: ion exchange, impregnation, incipient wetness, precipitation, physical or chemical adsorption, or co-precipitation. Metal catalysts that have been loaded on to these nanotubes include: ruthenium, osmium, rhodium, iridium, palladium, and platinum; the oxides, nitrides, phosphides, and sulfides of Ti, V, Cr, Mn, Fe, Co, Ni, Cu, Zn, Y, Zr, Mo, La, Ce, and W can also be loaded onto these nanotubes. This aggregated metal catalyst-functionalized carbon nanotube bundle can then be used to catalyze reactions. The advantages of using CNTs instead of conventional support is that CNTs have a large surface area, a greater number of mesopores (2-50 nm) and macropores (greater than 50 nm) than traditional carbon supports, and are resistant to attrition and catalyst deactivation.

U.S. Patent Application number 2010/0081726 discloses a micro-channel reactor optimized for a number of common reactions including the FT synthesis reaction. The patent discloses using a Co catalyst and, potentially, a co-catalyst from the group consisting of conventional FT synthesis catalysts such as Re, Cu, and Fe. The claimed process of conducting a FT reaction in these novel micro-channel reactors uses these conventional metal catalysts.

### SUMMARY OF THE INVENTION

One embodiment of this invention encompasses methods of utilizing highly-aligned multi-walled carbon and single-step metallocene-grown single walled carbon nanotubes as Fischer-Tropsch synthesis catalysts. This present technology as disclosed and claimed herein is the use of the catalytic properties of CNTs themselves and using these catalysts in a micro-channel reactor.

Another embodiment of the present invention encompasses methods of adjusting the reaction conditions of the Fischer-Tropsch synthesis reactions using these carbon nanotube catalysts in order to tune the hydrocarbon product distributions.

Other features and advantages of the invention will become manifest in the full embodiment of the invention.

### BRIEF DESCRIPTION OF DRAWINGS

FIG. 1 illustrates an SEM image of a MWNT bundle grown using the air-assisted vapor-phase catalyst delivery CVD method.

FIG. 2 illustrates a TEM image of a MWNT bundle grown using the air-assisted vapor-phase catalyst delivery CVD method.

FIGS. 3A-3C illustrates SEM images of the SWNT film produced by the direct decomposition of ferrocene at different magnifications.

FIG. 4 illustrates a TEM image of the SWNT film produced by the direct decomposition of ferrocene.

FIG. 5 illustrates Raman spectra obtained from the SWNT samples with pronounced RBM signatures.

FIG. 6 is a schematic of a fixed-bed FT reactor.

FIG. 7 illustrates a fractional distribution of the compounds obtained with respect to carbon number.

FIG. 8 illustrates TEM images of the as-produced 5-MWNTs and the purified 5-MWNTs.

FIG. 9 illustrates a comparison of the FT synthesis results on as-produced and purified 5-MWNTs.

FIG. 10 illustrates the effect of FT reaction temperature on the products using 5-MWNTs as the catalyst and 3:1 syngas ratio.

FIG. 11 illustrates the effect of FT reaction temperature on the products using 5-MWNTs as the catalyst and 1:1 syngas ratio.

FIG. 12 illustrates the effect of FT reactor pressure on the products using 5-MWNTs as the catalyst and 1:1 syngas ratio.

FIG. 13 illustrates the liquid product distribution of different MWNTs at 300 C.

FIG. 14 illustrates the liquid product distribution of different MWNTs at 350 C.

FIG. 15 illustrates the liquid product distribution of different MWNTs at 400 C.

FIGS. 16A-16D illustrate (a) Product distribution obtained with (b) MWNTs grown on coerdite monoliths.

FIG. 17 illustrates the setup used to verify the ability of the CNTs to convert syngas into liquid hydrocarbon fuels.

FIG. 18 illustrates the process by which CNTs convert syngas into liquid hydrocarbon fuels.

### DETAILED DESCRIPTION

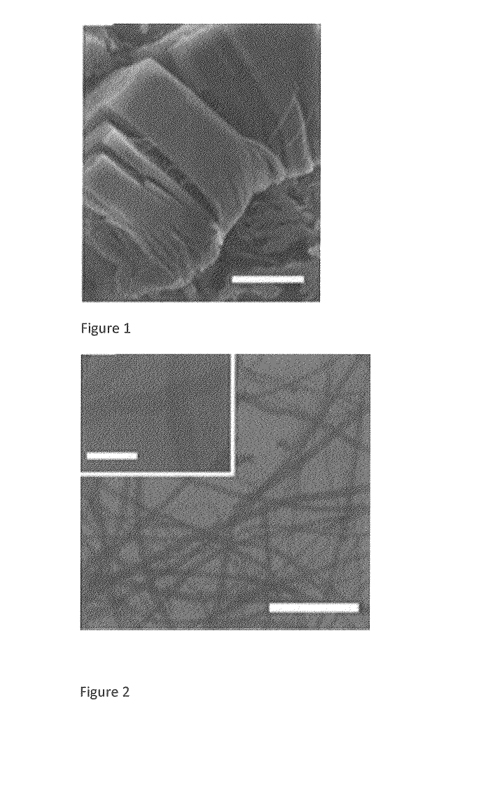

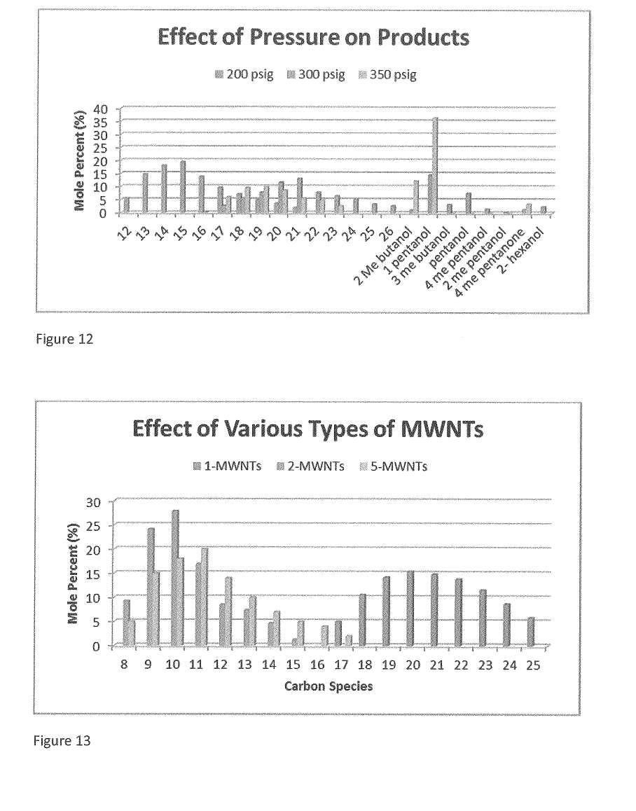

According to the embodiment(s) of the present invention, various views are illustrated in FIGS. 1-16 and like reference numerals are being used consistently throughout to refer to like and corresponding parts of the invention for all of the various views and figures of the drawing. Also, please note that the first digit(s) of the reference number for a given item or part of the invention should correspond to the Fig. number in which the item or part is first identified.

One embodiment of the present technology comprising a method of utilizing highly-aligned multi-walled carbon and single-step metallocene-grown single walled carbon nanotubes as Fischer-Tropsch synthesis catalysts which teaches a novel method for producing liquid fuels. The details of the invention and various embodiments can be better understood by referring to the figures of the drawing.

Synthesis of Aligned MWNTs Using Air-Assisted Vapor Phase Catalyst Delivery.

The technology as disclosed and claimed herein relates to the implementation of an air assisted vapor phase catalyst delivery CVD method for growing CNTs. These multi-walled CNTs were produced using the general procedures outlined in X. Li, X. Zhang, L. Ci, R. Shah, C. Wolfe, S. Kar, S. Talapatra and P. M. Ajayan, “Air assisted growth of long aligned carbon nanotube films”, Nanotechnology 19, 455609 (7 pp) (2008) which is herein incorporated by reference.

In order to grow vertically aligned multi-walled CNTs, a metal-organic complex, preferably a Group VIIB metallocene such as cobaltocene or ferrocene, may be combined with an easily vaporized carbon source. This carbon source can be short-chain hydrocarbons such as methane, ethane, propane, ethylene or acetylene or an aromatic hydrocarbon species such as xylene, benzene, cumene, toluene, ethylbenzene, naphthalene, or anthracene. A solution of the metallocene and the carbon source may be vaporized and flown into a chemical vapor deposition furnace. The furnace temperature is carefully calibrated so that the catalyst particles precipitate on the substrate from the vapor and the carbon atoms dissociate from the carbon source. One advantage of the air-assisted vapor delivery method of growing carbon nanotubes is that large amounts of MWNTs will grow on relatively irregular substrates. Therefore, a substrate is selected that is relatively resistant to the ambient temperatures of the furnace and is relatively smooth; many such substrates are recognized in the art and include a substrate composed of such common materials as: silicon, Inconel, quartz, or ceramics.

In one implementation of the technology, gram-quantities of highly aligned MWNTs using ferrocene as the metallocene and xylene as the carbon source can be grown. Three different ferrocene-xylene solutions consisting of 1.0, 2.0, or 5.0 grams of ferrocene to 100 mL of xylene can be made. 15 mL of each of the three ferrocene solutions can be vaporized in a steel bottle and flown into a tube-style CVD furnace containing substrates for CNT growth. Argon can be used as an inert gas to purge and equilibrate the furnace and a mixture of Argon/Hydrogen (˜85% Ar/15% H₂) can be used as the carrier gas. The furnace can be carefully calibrated to a temperature of ˜790° C. After the CVD furnace system reaches steady-state, the carrier gas can be pumped into the system at a rate of ˜12 mL/hr and the ferrocene/xylene solutions can be carried into the reaction zone of the furnace by the carrier gas. After an adequate deposition time, the inlet gas can be switched back to the carrier gas while the furnace cooled. The substrate and nanotube sample can be removed from the reaction zone and the nanotube can be peeled or scraped from the surface. Images of the aligned MWNTs created using this method were obtained using Scanning electron microscopy (SEM); an SEM image is shown in FIG. 1. A Transmission Electron Microscopy (TEM) image of the MWNTs grown using this process is shown in FIG. 2.

Synthesis of SWNTs Using Direct Decomposition of Ferrocene

The synthesis of carbon nanotubes (CNTs) by direct thermal decomposition of a metallocene is a single step production method to obtain SWNT macro-films with controllable dimensions and high metal content.

The metallocene acts as both the catalyst and the carbon source; any Group VIIB metallocene such as cobaltocene or ferrocene can be used. Sulfur is added to the metallocene to promote growth of SWNTs and is placed near the heat source in a long, large-diameter tube furnace. The use of a tube furnace serves dual purposes: it not only acts as the furnace but as the substrate for CNT deposition. A carrier gas is flown into the reaction tube to allow for the metallocene vapors to reach the ends of the tubes and allow for CNT growth in the cool ends of the tube. The flow rate of buffer gas, furnace temperature, and sulfur/metallocene concentration are the factors that promote growth rate and concentration of SWNTs.

In one implementation of the technology disclosed and claimed herein, SWNT growth can be carried out in a horizontal tube furnace. A ceramic boat (approximately 8 cm long) containing a ferrocene/sulfur mixture (at about a 10:1 ratio) was placed at one end of the quartz tube approximately 2 feet away from the center of the furnace. The set point of the furnace can be adjusted so that the middle of the furnace containing the quartz tube reaches 1150° C. The temperature at of the quartz tube end containing the ferrocene/sulfur mixture can be then monitored using a thermocouple. When the temperature of the quartz tube in the region containing the ferrocene/sulfur mixture reaches ˜350° C.-400° C., the mixture starts to evaporate. At this stage a mixture of Ar and H2 gas (˜Ar 85%/H₂ 15%) is introduced into the quartz tube at a rate of about 2000 seem to carry the ferrocene vapors into the reaction zone (middle of the furnace). The CNTs are produced from the ferrocene vapors in the reaction zone is carried by the carrier gas and gets deposited on the walls of the quartz tubes as macroscopic films on the other end of the quartz tube (the tube exhaust).

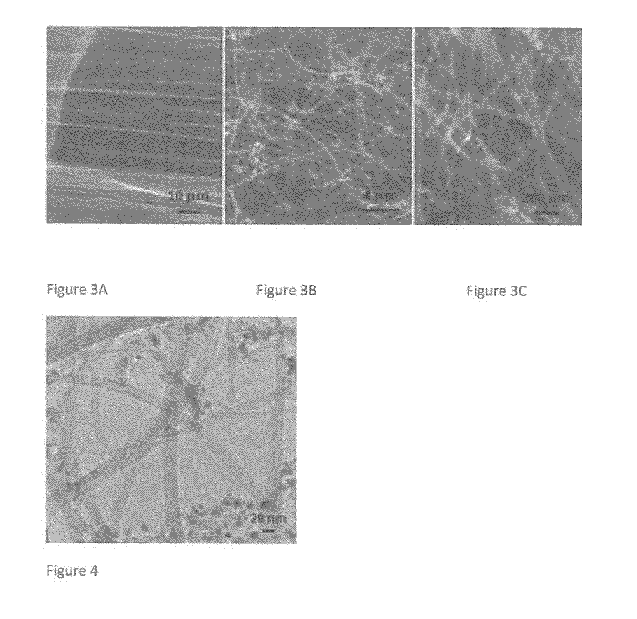

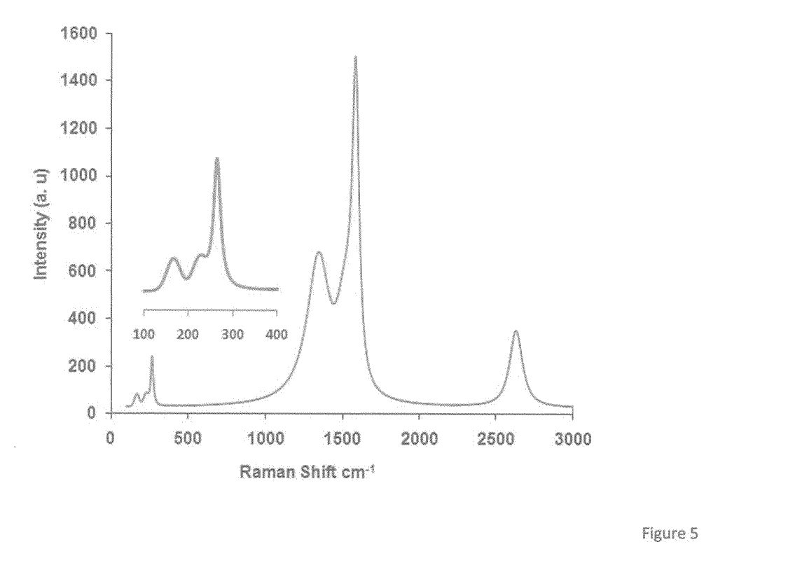

The CNTs produced by this method were then peeled from the reaction tube and analyzed using a FEI Quanta™ 450 FEGScanning Electron microscope. In FIG. 3, electron microscopy images of some of the as-produced CNT films are presented. A visual inspection of the films indicated that the CNTs were assembled as thick macroscopic films, however it was possible to peel off very flexible transparent films containing thin diameter CNTs from the CNT macro film, as shown in the SEM image FIG. 3a. FIGS. 3b and 3c shows higher magnification images of the CNT films. These images clearly indicate the presence of thin diameter CNTs present in the sample. A TEM image of this sample is shown in FIG. 4. From the TEM image, it can be seen that the as-grown samples consisted of ropes of SWNTs.

To obtain a better estimate regarding the quality of the CNTs produced in this process the samples were characterized using Raman spectroscopy, a standard technique for non-destructive characterization of carbon materials. The first order Raman spectral features in the higher frequency region (wavenumbers>1200 cm-1), showed several strong peaks between about 1300 cm-1 and 1600 cm-1 (FIG. 5). The peak centered around ˜1350 cm-1 corresponds to the disorder induced, D-band of graphite and is due to the presence of amorphous carbon, defects in nanotubes and other carbon materials present in the sample. Along with this peak, another strong peak with values close to that of the G band (corresponding to the graphite E2g optic mode) of graphite at ˜1582 cm-1 was also observed in the sample. This mode is due to the in-plane bond stretching motion of pairs of sp2 carbon atoms and can lie anywhere between about 1500 cm-1 and 1630 cm-1 and therefore, the peaks within this range is assigned to be the G band of graphite. This window of wavenumbers is also very crucial in determining the morphology of the CNTs, since it has been demonstrated that the Raman frequencies of SWNT and/or thin diameter nanotubes, specifically within this wavenumber window, are substantially different from MWNTs. For example if we consider the Raman spectra of the sample, we can observe two peaks in this region. One sharp peak ˜1598 cm-1 and another prominent peak ˜1560 cm-1, up-shifted and downshifted respectively from the regular E2g (1582 cm-1) mode of graphite. This is a direct consequence of the zone-folding, which lifts the degeneracy of the E2g mode in CNTs, resulting in a spread in the mode frequencies with decreasing CNT diameters.

Another distinct feature that can be observed from the Raman data is the presence of several peaks at the low frequency region. Typically a strong feature around ˜186 cm-1 is present in SWNTs and is assigned to A1g breathing mode (radial breathing mode RBM). The RBM frequencies are sensitive to the diameters of the nanotubes and their appearance in the Raman spectra indicates the presence of SWNT and/or thin diameter MWNTs.

CNTs as Fischer-Tropsch Synthesis Catalysts

The advantage of using either the MWNTs produced from the reaction of a carbon source with a metallocene catalyst or the SWNTs produced from a metallocene is that they can be used as catalysts as is. That is, they can be loaded directly into any typical Fischer Tropsch reactor, including but not limited to fixed bed reactors, fluidized bed reactors, or slurry bed reactors, without impregnating them with any conventional catalyst. In fact, removing the residual metal particles that naturally adhere to the surface during the CNT growth process only improves the catalytic efficiency. The prior art only seems to recognize these CNTs as a support material rather than as an efficient catalyst. These CNTs can either be loaded into the reactor directly or through attachment to an inert support material such as silicon, Inconel, quartz, or ceramics to allow for ease of use.

Experiment 1: Fischer-Tropsch Synthesis Experiments.

The CNTs produced using the methods detailed above were used as FT synthesis catalysts, without any further purification or alterations. These as-produced CNTs were compared to the conventional catalyst of Fe—Zn—K disposed on a γ-Al₂O₃ support. FT synthesis were conducted in a flat-bed reactor at various temperatures using the conventional catalyst as the control and the as-produced aligned multi-walled (ferrocene/xylene) and single-walled (single-step production from ferrocene) CNTs with no further purification.

The conventional catalyst Fe—Zn—K disposed on a γ-Al₂O₃ support was synthesized via the precipitation and impregnation synthesis method well-known in the art for synthesizing this type of catalyst as discussed in published U.S. Pat. No. 7,365,040 which is herein incorporated by reference. A pre-formed γ-Al₂O₃ substrate was purchased from AlfaAesar (CAS 1344-28-1). The support (20 g γ-Al₂O₃) of was impregnated with Zn and Fe through immersion in a slurry of Fe(NO₃)₂ (3M) and Zn(NO₃)₂ (1M) for 10 hours. The impregnated support was then subject to two drying steps: ˜12 hours in a ˜100° C. oven and ˜16 hours in a ˜350° C. oven. Then the support was further impregnated with K in a 0.16 M K₂CO₃ solution for one hour. The K impregnated support was then further dried for ˜15 hours in a ˜100° C. oven and for ˜4 hours in a ˜450° C. oven

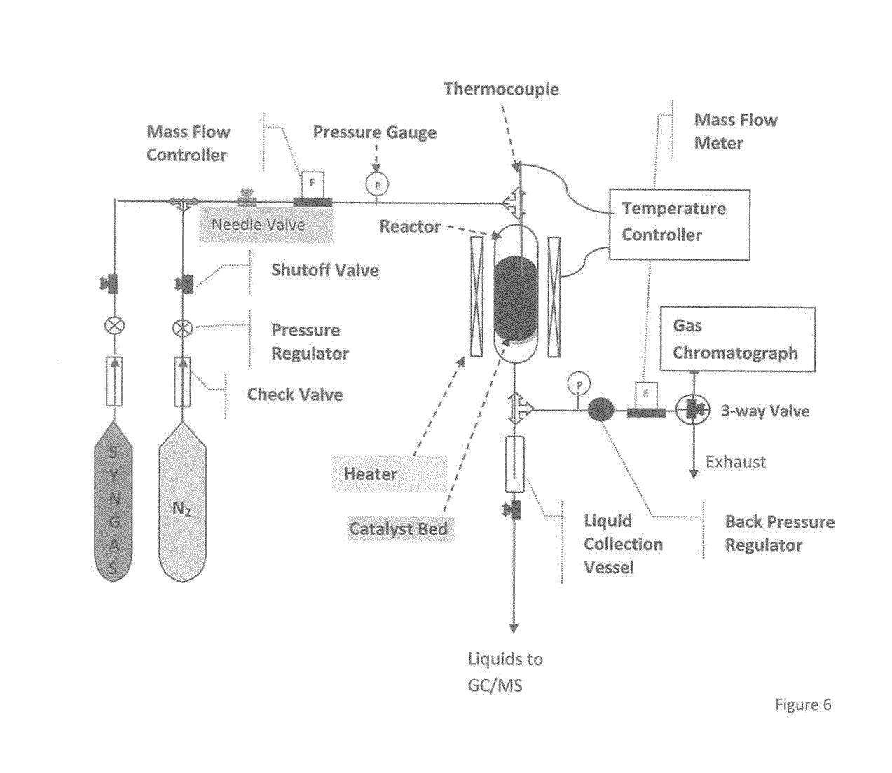

Approximately 1.0 gram of the conventional catalyst was massed and loaded into the fixed bed reactor with a continuous flow design as shown in FIG. 6. The syn-gas ratio was 1:1 (mol H₂: mol CO) and at both ˜300° C. and ˜350° C. The residence time of the syn-gas mixture on the conventional catalyst was ˜2 hours at ˜300 psig. This experimental protocol was reproduced for both the as-produced MWNTs and SWNTs. The liquid products were collected and were analyzed using Gas Chromatography for liquid product distribution. The outlet gas from each Fischer-Tropsch reaction was redirected to flow through a Buck Scientific (model 910) Gas Chromatograph to be analyzed for CO content. Then the % conversion of syn-gas mixture to hydrocarbon products was measured using the following equation:

$$Conversion \% = \frac{CO {\%}_{inlet} - CO {\%}_{outlet}}{CO {\%}_{inlet}} \times 100.$$

TABLE 1

Conversion (%)/
gm of catalyst

| Catalyst | T (° C.) | CO | H₂ |
|---|---|---|---|
| Fe—Zn—K/γAl₂O₃ | 300 | 2.89 | 2.92 |
| Aligned MWNT | 300 | 132.98 | 145.41 |
| Ferrocene derived SWNT | 300 | 317.27 | 316.69 |
| Fe—Zn—K/γAl₂O₃ | 350 | 6.45 | 8.19 |
| Aligned MWNT | 350 | 156.91 | 168.44 |
| Ferrocene derived SWNT | 350 | 368.06 | 313.13 |

Then Fe content contained in the conventional catalyst, the as-produced SWNTs, and the as-produced MWNTs was measured using both energy dispersive spectroscopy and thermal gravimetric analysis and compared to the % conversion of syn-gas mixture.

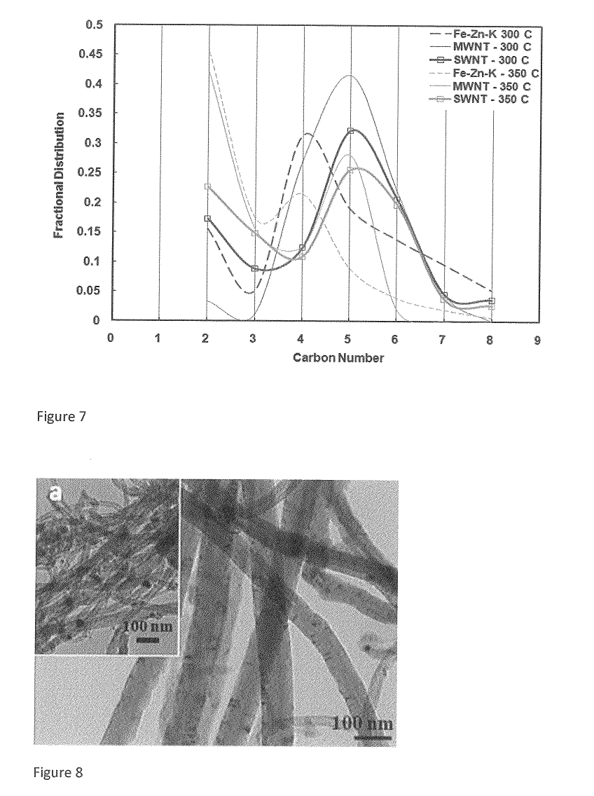

As shown in Table 1, the as-produced CNTs show at least an order of magnitude improvement in % conversion of reactants/gm of catalyst when compared to the conventional Fe—Zn—K/γ-Al₂O₃ catalyst. Additionally, these CNTs produced hydrocarbons with greater carbon numbers than the conventional catalyst. FIG. 7 shows the fractional distribution of the hydrocarbons produced from the FT synthesis reactions (at ˜300° C. and ˜350° C.) catalyzed with the as-produced MWNTs, SWNTs, and the conventional catalyst. The as-produced MWNTs and SWNTs showed an order of magnitude improvement in conversion efficiency (Conversion %/gm of catalyst) over the conventional catalyst with a tendency to produce hydrocarbons with larger carbon numbers. Although these results were encouraging, at the time it was unclear whether the reaction was being catalyzed by the CNTs themselves or by the iron particles attached to the CNTs. Although the Fe content in the as-produced MWNTs was only about 0.014 gm and only 0.04 gm in the as-produced SWNTs (compared to the 2.5 gm Fe content in the conventional catalyst based on 12.5% Fe content and 20 grams of catalyst), it was still unclear whether it was the Fe adhered to the surface of the CNTs catalyzing the reaction or the CNTs themselves.

Experiment 2—Fischer-Tropsch Synthesis on Purified MWNTs

In order to elucidate whether it was the Fe adhered to the surface of the CNTs catalyzing the reaction or the CNTs themselves, the Fe particles were removed from the surface of the as-produced CNTs through a purification process.

Materials and Methods:

The MWNTs produced from a ratio of 5 g Ferrocene to 100 mL xylene (5-MWNTs) employing the methods discussed above were purified using acid treatment. Approximately 0.8 g sample of the as-produced 5-MWNTs were obtained and baked in a 400° C. oven for one hour to remove any trace amorphous carbon. Then these 5-MWNTs were immersed in 50 ml of hydrogen peroxide (H₂O₂ 30 vol %) solution. The MWNTs in the solution was then dispersed using ultrasonic agitation for about 30 mins. After the sonication the MWNTs were left in the solution for 24 hrs. Subsequently 50 ml of hydrochloric acid (HC137 vol %) was slowly added into this the solution containing CNTs and H₂O₂. This mixture was left to stand for several hours. Finally 100 ml of DI water was added into the solution and vacuum filtered through a porous membrane to obtain a mat of purified 5-MWNTs. TEM images of the purified CNTs were obtained using a Hitachi 7100.

The acid-digested 5-MWNT catalyst sample was then loaded into the a fixed bed reactor with a continuous flow design as shown in FIG. 6 to be used as a catalyst in a FT synthesis reaction. The FT synthesis reaction conditions in this experiment were identical to those contained in Experiment 1, above. The syn-gas ratio was 1:1 (mol H₂: mol CO) and at both ˜300° C. and ˜350° C. The residence time of the syn-gas mixture on the conventional catalyst was 2 hours at 300 prig. The liquid products were collected and were analyzed using Gas Chromatography for liquid product distribution. The outlet gas from each Fischer-Tropsch reaction was redirected to flow through a Buck Scientific (model 910) Gas Chromatograph to be analyzed for both CO and H₂ content.

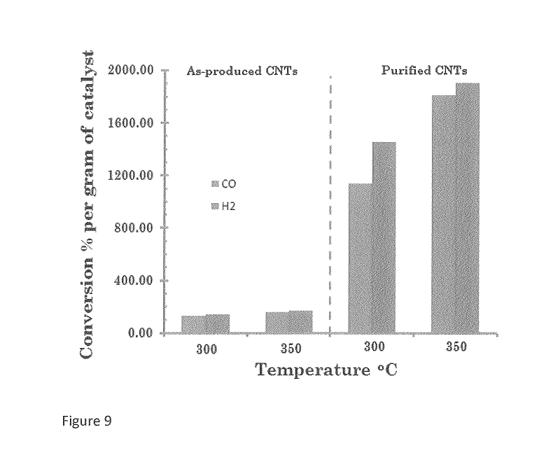

The purification process was effective at removing the metal impurities. TEM images of the as-produced 5-MWNTs (FIG. 8 (inset) and the purified 5-MWNTs (FIG. 8) indicate the efficacy of this purification process. Initially, the conversion percentage of the purified MWNTs was expected to be lower than the as-produced MWNTs; however, the conversion percentage in the FT synthesis reaction using the purified 5-MWNTs was two orders of magnitude higher than the as-produced 5-MWNTs (FIG. 9.) Therefore, from this increase in conversion efficiency of the purified 5-MWNT catalyzed FT reaction despite the reduced iron content of the catalyst, we can conclude that it is the nanotube structure that is encouraging the formation of these hydrocarbon products rather than the availability of the metal catalyst particles to the syngas caused by the high surface area of the CNTs.

Experiment 3—Tuning Fischer-Tropsch Product Distribution Using Reaction Temperature in CNT Catalyzed Reaction

Materials and Methods:

The as-produced 5-MWNTs were tested in the continuous-flow fixed-bed FT reactor shown in FIG. 6. A syngas ratio of 3:1 (moles H₂: moles CO) at 300 psig with a syngas residence time of 2 hours at each of the following temperatures: 200, 300, 350 and 400° C. The liquid products were collected and analyzed using GC for liquid product distribution. The outlet gas from each Fischer-Tropsch reaction was redirected to flow through a Buck Scientific (model 910) Gas Chromatograph to be analyzed for both CO and H₂ content. N₂ gas was flown into the reactor bed during temperature adjustments to purge the reactor.

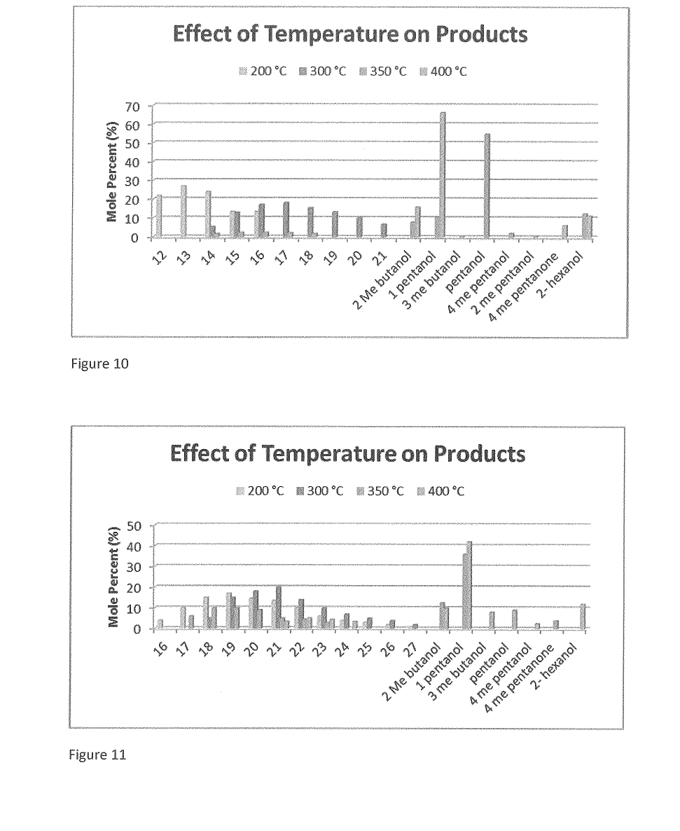

The effect of temperature change on the product distribution is shown in FIG. 10. At ˜200 and ˜300° C. the primary products were hydrocarbons ranging from C₁₂-C₁₆, which is ideal for jet fuel. Increasing the temperature from ˜200 to ˜300° C. promoted chain growth, resulting in more near the C₁₇ range (up from the C₁₃ range), a distribution typically observed in diesel fuels. As the temperature was furthered increased to ˜350 and ˜400° C., there was a sharp change in products toward the formation of alcohols, mostly pentanol (over 50%). The experiment conducted at ˜400° C. did not result in any hydrocarbons. However, the product distribution at this temperature also showed ketones, namely pentanone (≈5%). From this, we conclude that these MWNTs show excellent conversion efficiency, as well as a tunable product distribution as FT catalysts.

Experiment 4—Effect of Syn-Gas Ratio on Fischer-Tropsch Product Distribution

Materials and Methods:

The as-produced 5-MWNTs were tested in the continuous-flow fixed-bed FT reactor shown in FIG. 6. All of the parameters of Experiment 3, above, were used except the syngas ratio was 1:1 (moles H₂: moles CO.) The syngas residence time was ˜2 hours at ˜300 psig at each of the following temperatures: ˜200, ˜300, ˜350 and ˜400° C. The liquid products were collected and analyzed using GC for liquid product distribution. The outlet gas from each Fischer-Tropsch reaction was redirected to flow through a Buck Scientific (model 910) Gas Chromatograph to be analyzed for both CO and H₂ content. N₂ gas was flown into the reactor bed during temperature adjustments to purge the reactor.

The product distribution obtained with a 1:1 syn-gas ratio is shown in FIG. 11. Similar results were obtained with a 1:1 syn-gas ratio as with a 3:1 syngas ratio (Experiment 3, above). Experiments at ˜200 and ˜300° C. still produced hydrocarbons of medium carbon chain lengths, and slightly longer chains were realized with the increase in temperature. At ˜200° C., alkanes with chain lengths ranging from C₁₆ to C₂₇ with a peak concentration around C₁₈-C₂₁ were produced. Upon increasing the temperature to ˜300° C., the products contained alkanes with carbon chain lengths from C₁₈-C₂₇ with the peak concentration in the range of C₁₉-C₂₂. At ˜350 and ˜400° C., pentanol was still shown to represent an overwhelming majority of the products; however, there were also more hydrocarbons in the C₁₉-C₂₀ range (at ˜350° C.) and C₂₂-C₂₃ range (at ˜400° C.) with the 1:1 syngas ratio.

Experiment 5—Effects of Pressure on Fischer-Tropsch Product Distribution

Materials and Methods:

The as-produced 5-MWNTs were tested in the continuous-flow fixed-bed FT reactor shown in FIG. 6. A syngas ratio of 1:1 (moles H₂: moles CO) at ˜350° C. with a syngas residence time of ˜2 hours at each of the following pressures: ˜200, ˜300, and 350˜psig. The liquid products were collected and analyzed using GC for liquid product distribution. The outlet gas from each Fischer-Tropsch reaction was redirected to flow through a Buck Scientific (model 910) Gas Chromatograph to be analyzed for both CO and H₂ content.

Results as shown in Figure, 12 an increase in pressure from ˜200 psig to ˜300 psig resulted in a significant increase in hydrocarbon chain growth, from C₁₅ to C₂₁ peak length, but also resulted in the formation of alcohols. At ˜350 psig, there was little change in the hydrocarbon distribution but fewer total hydrocarbons produced, as the yield comprised over 50% alcohols at that temperature. From this we can conclude that higher pressures have an affinity for alcohol production with this temperature and syngas ratio.

Experiment 6—Effect of Ferrocene: Xylene Ratio on MWNT Catalyzed Fischer-Tropsch Reactions

Materials and Methods:

In one implementation for growing MWNTs, discussed above, we grew highly aligned MWNTs using ferrocene as the metallocene and xylene as the carbon source. Three different ferrocene-xylene solutions consisting of 1.0, 2.0, or 5.0 grams of ferrocene to 100 mL of xylene were made. The MWNTs grown using this process are referred to as 1-MWNTs, 2-MWNTs, and 5-MWNTs, respectively. Approximately 1.0 grams of the as-produced 1-MWNTs, 2-MWNTs, and 5-MWNTs were tested in the continuous-flow fixed-bed FT reactor shown in FIG. 6. A syngas ratio of 1:1 (moles H₂: moles CO) at ˜300 psig with a syngas residence time of ˜2 hours at each of the following temperatures: ˜300, ˜350 and ˜400° C. The liquid products were collected and analyzed using GC for liquid product distribution. The outlet gas from each Fischer-Tropsch reaction was redirected to flow through a Buck Scientific (model 910) Gas Chromatograph to be analyzed for both CO and H₂ content. N₂ gas was flown into the reactor bed during temperature adjustments to purge the reactor.

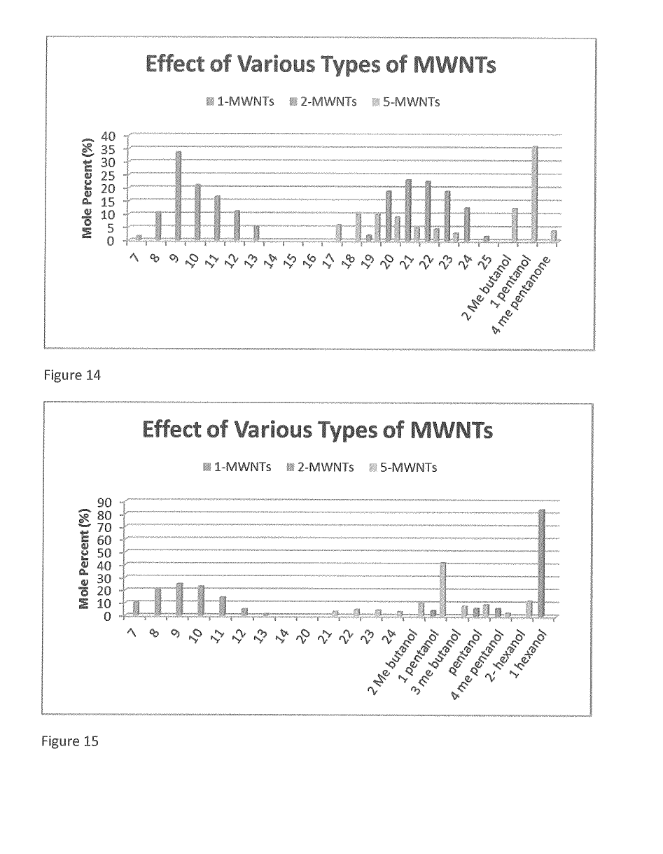

The liquid products distribution based on carbon number is shown for 1-MWNTs, 2-MWNTs, and 5-MWNTs at ˜300° C. (FIG. 13), ˜350° C. (FIG. 14), and ˜400° C. (FIG. 15.) The 2-MWNTs and 5-MWNTs had a peak hydrocarbon chain length of C₁₀ and C₁₁ at ˜300° C. The 1-MWNTs favored chain growth with peaks in the C₂₀-C₂₁ range. At ˜350° C., both the 1-MWNTs and 2-MWNTs produced mostly hydrocarbons, but of different lengths. The product distribution from the 1-MWNTs shows carbon chain lengths in the C₂₀-C₂₄ range, while the 2-MWNTs produced much shorter hydrocarbons in the C₉-C₁₂ range. With the 5-MWNTs, the hydrocarbon chains were in the C₁₉ range but a substantial percent of the yield was found to be alcohols.

The products of the 2-MWNT sample were similar at ˜400° C. as well. The results for the 5-MWNTs are also similar to those at ˜350° C., with pentanol making up over 35% of the products and the few hydrocarbons around the C₂₂ range. At this temperature, though, the 1-MWNTs had a drastic change in results, producing over 80% hexanol and no hydrocarbons. Besides the increase in alcohol formation at higher temperatures, no clear correlation between MWNT type and product distribution can be determined.

Experiment 7: FT Synthesis of CNTs Loaded on Cordierite Monoliths

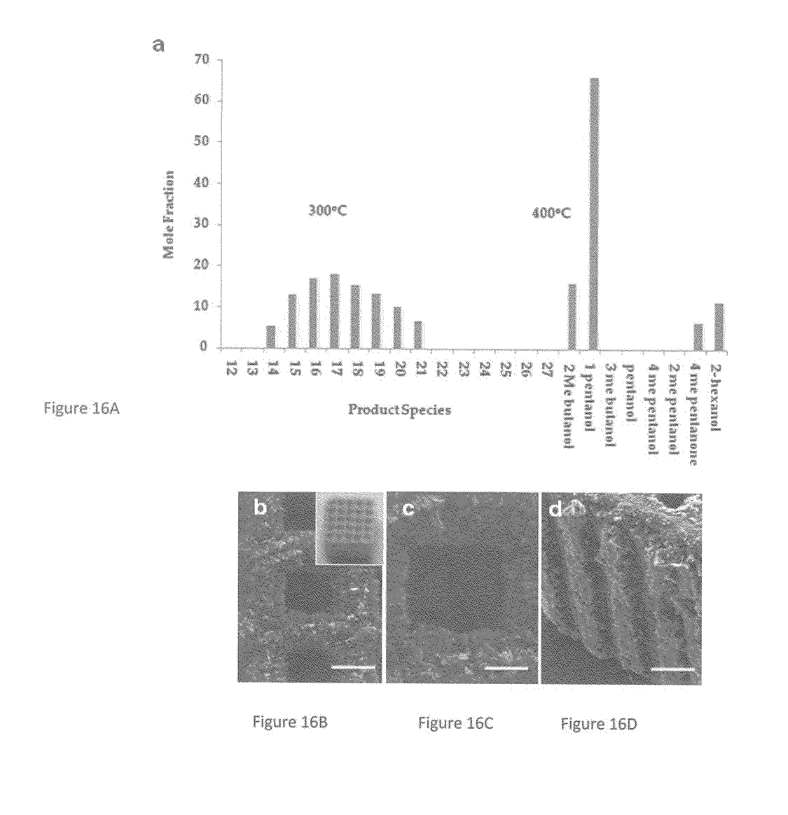

In order to facilitate the use of these CNT catalysts in the industry, the CNTs were loaded onto commercial supports such as cordierite in a single step and the as produced cordierite supported CNT was used for FT synthesis. FIG. 16 shows the catalyst and the products obtained.

Materials and Methods:

Air assisted vapour phase catalyst delivery CVD method for growing CNTs on cordierite monoliths. A solution of ferrocene-xylene (5 g ferrocene/100 mL xylene) was vaporized in a steel bottle and was flown into a furnace (using Argon/Hydrogen (˜85% Ar)) containing the substrates (monoliths in this case) for CNT growth. As produced CNTs grown on the substrate was used as catalyst in the reactor for performing the FT synthesis. FIG. 16 b shows CNTs grown directly on as received cordierite monoliths (inset) and FIG. 16 c and d scanning electron microscopy images of different portions of the coerdite monolith showing uniform growth of CNT.

The cordierite supported CNT sample was then loaded into the a fixed bed reactor with a continuous flow design as shown in FIG. 6 to be used as a catalyst in a FT synthesis reaction. The syn-gas ratio was 3:1 (mol H₂: mol CO) and at both ˜300° C. and ˜400° C. The syngas flow rate was ˜20 standard mL/min. The liquid products were collected and were analyzed using Gas Chromatography for liquid product distribution. The outlet gas from each Fischer-Tropsch reaction was redirected to flow through a Buck Scientific (model 910) Gas Chromatograph to be analyzed for both CO and H₂ content. FIG. 16a shows product distribution obtained with respect to reaction temperature using 3:1 ratios of CO:H₂ on CNT supported by cordierite. The ability to load the CNTs onto commercial supports and the ability of the catalyst to convert syngas to liquid organics is demonstrated in the figure.

Therefore, to reiterate what was discussed above, converting carbonaceous materials such as coal, biomass, natural gas to liquid fuels such as gasoline, diesel, jet fuel, kerosene etc is generally accomplished through three process blocks. The first block (generally referred to as the gasification/reforming block) consists of operations that would convert the carbonaceous materials to syngas by reacting it with steam or oxygen (less than stoichiometric requirement) or both. The product from this block is a mixture of carbon monoxide (CO) and hydrogen (H₂) commonly referred to as synthesis gas or syngas. The first bock also often consists of gas cleanup operations to remove impurities and other co-products. The second block primarily deals with removing the 0 from the CO in syngas using the H₂ in the syngas and add hydrogen to the C formed to form carbene (—CH₂—) moieties and water. In the same reactor, the —CH₂-moieties polymerize to form longer chains (—CH₂—CH₂—CH₂- . . . -CH₂—). Depending of the length of the hydrocarbons formed, they can be classified into different liquid transportation fuels we know. Thus, essentially, CO hydro-deoxygenation followed by hydrocarbon chain growth process can convert syngas (mixture of CO and H₂) to liquid hydrocarbon fuels. It is this block for which the present technology as disclosed and claimed herein addresses. The most well-known process for achieving the above is the Fischer Tropsch Synthesis (FTS) process catalyzed by the late transition metal catalysts such as Fe, Co, Ru. In this patent, the present technology as disclosed and claimed herein leverages the use of a particular form of carbon—carbon nanotubes—as the catalyst themselves. The present technology in its various implementations shows that the CNTs produced via air-assisted chemical vapor deposition (CVD) can produce these extremely active catalysts (over an order higher in the magnitude of the activity of the catalyst as compared to the conventional Fe based catalyst) in a single step.

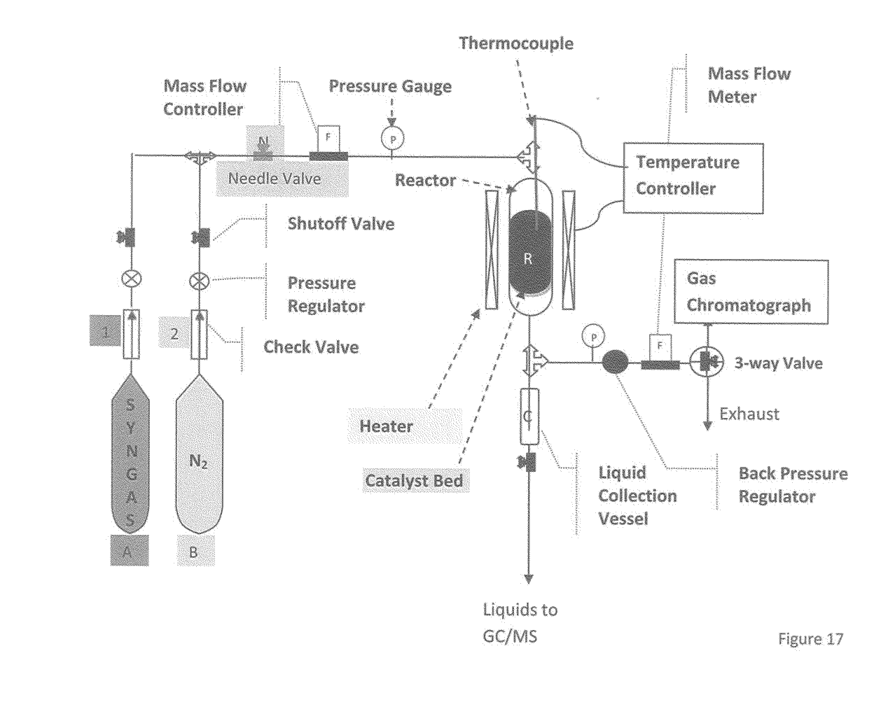

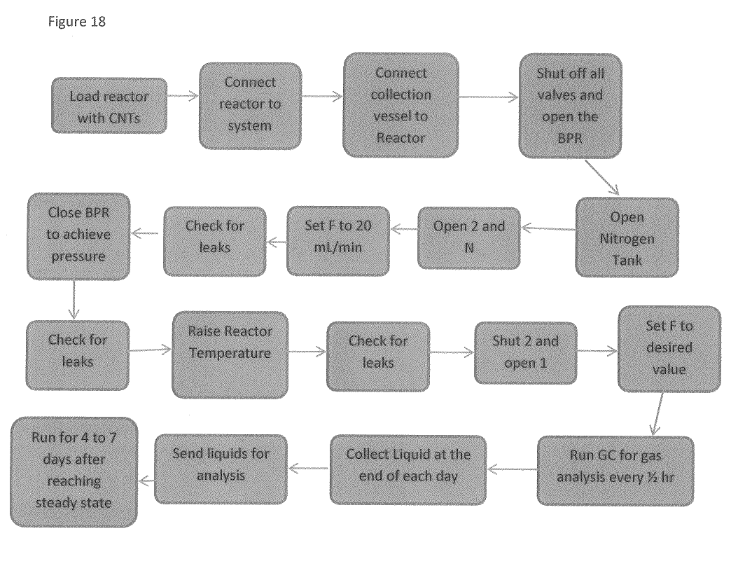

The setup used to verify the ability of the CNTs to convert syngas into liquid hydrocarbon fuels is shown in FIG. 17. The method can be conducted in a continuous flow reactor (R) in which the CNTs (<0.5 g) were packed. The apparatus accommodates two inlets from two gas tanks (Syngas-A or Nitrogen-B) converging into a single line that leads to the reactor. The nitrogen is used for flushing all the air from the reactor and for pressurizing the system and ensuring that there are no leaks (dry run). Once the safety issues have been verified and the reactor temperature can be raised to the desired value, shutoff valve 2 can then be closed while shut off valve 1 (for syngas) is opened. There can be an additional shut off valve, N, for additional safety. This valve is normally open but is closed in case of leaks or temperature runaway. A mass flow controller, F, (such as Brook's instrument) can be placed before the reactor which helps to maintain the feed gas flow rate. The reactor (R) in one implementation can have a 16 inch long and ½ inch diameter tubular reactor wrapped by an electric heating tape (such as OMEGALUX flexible heating tapes). The heating tape was connected to the temperature controller (Omega csi8D series) and Omega K-type thermocouple (whose tip was placed inside the reaction zone) to maintain the temperature in the reaction zone. The products leaving the reactor chamber can be passed through a condenser (C) where the condensable products (water and normally liquid hydrocarbons) are collected while the gases leave the condenser and flow through a back pressure regulator (BPR) to a gas chromatograph (GC) (Agilent technology, 490 micro GC). Liquid collecting tube can be attached to shutoff valve at end and can be submersed in a container of ice cold water. The connecting line can be covered with ice bag. Flow meter can be placed next to pressure regulator to read final gas flow rate. Then the gas reaches a two way shut-off valve. Among them one is directed to fume hood whereas other to the GC for gas spectrum analysis (used when gas analysis is planned). The liquids were sent out for analysis to an external laboratory. The flow diagram shown in FIG. 18 is illustrates one implementation of the process.

The various implementation of the carbon nanotube process shown above illustrates a novel one step process for the use of CNTs produced via air-assisted chemical vapor deposition (CVD) which can produce these extremely active catalysts (over an order higher in the magnitude of the activity of the catalyst as compared to the conventional Fe based catalyst) in a single step. A user of the present technology may choose any of the above implementations, or an equivalent thereof, depending upon the desired application. In this regard, it is recognized that various forms of the subject process and system could be utilized without departing from the spirit and scope of the present invention.

As is evident from the foregoing description, certain aspects of the present invention are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the spirit and scope of the present invention.

What is claimed:

1. A method for converting carbonaceous materials to liquid fuels comprising the steps of:
receiving at a reactor tube, having a carbon nano-tube, synthesis gas from a gasification/reforming step of a liquid fuel production process, where the carbon nano-tube is produced by an air-assisted chemical vapor deposition thereby having attached oxygen functional groups;
reacting the carbon monoxide of the synthesis gas with the surface of the carbon nano-tube where the oxygen functional groups act as a catalyst thereby removing oxygen from the carbon monoxide using hydrogen in the synthesis gas;
adding hydrogen to the carbon to form carbene moieties and water; and
polymerizing the carbene to form longer hydrocarbon chains.

2. The method as recited in claim 1, where the carbon nano-tube is a multi-walled nano-tube produced from the reaction of a carbon source with a metallocene catalyst.

3. The method as recited in claim 2, where the multi-walled nano-tube has been purged of metal particles that naturally adhere to the surface of the carbon nano-tube during the process of growing the carbon nano-tube.

4. The method as recited in claim 1, where the carbon nano-tube is a single-walled nano-tube produced from a metallocene.

5. The method as recited in claim 4, where the single-walled nano-tube has been purged of metal particles that naturally adhere to the surface of the carbon nano-tube during the process of growing the carbon nano-tube.

6. A system for converting carbonaceous materials to liquid fuels comprising:
a reactor tube, having a carbon nano-tube, downstream from a gasification/reforming system block of a liquid fuel production system which provides synthesis gas to the reactor, where the carbon nano-tube is produced by an air-assisted chemical vapor deposition having oxygen functional groups attached to the surface of the carbon nano-tube;
a flow of synthesis gas in the reactor tube reacting the carbon monoxide of the synthesis gas with the surface of the carbon nano-tube where the oxygen functional groups act as a catalyst thereby removing oxygen from the carbon monoxide using hydrogen in the synthesis gas;
carbene moieties and water formed in the reactor from hydrogen added to the carbon; and
polymerized carbene forming longer hydrocarbon chains.

7. The method as recited in claim 6, where the carbon nano-tube is a multi-walled nano-tube produced from the reaction of a carbon source with a metallocene catalyst.

8. The method as recited in claim 7, where the multi-walled nano-tube has been purged of metal particles that naturally adhere to the surface of the carbon nano-tube during the process of growing the carbon nano-tube.

9. The method as recited in claim 6, where the carbon nano-tube is a single-walled nano-tube produced from a metallocene.

10. The method as recited in claim 9, where the single-walled nano-tube has been purged of metal particles that naturally adhere to the surface of the carbon nano-tube during the process of growing the carbon nano-tube.
